(19) United States
(12) Patent Application Publication
(10) Pub. No.: US 2008/0015553 A1
(43) Pub. Date: Jan. 17, 2008

(54) Steering laser treatment system and method of use

(76) Inventor: Jaime Zacharias, (US)

Correspondence Address: JAIME ZACHARIAS, AV. LUIS PASTEUR 5917 - VITACURA, SANTIAGO, 6670775, omitted

(21) Appl. No.: 11/456,863

(22) Filed: Jul. 12, 2006

(51) Int. Cl. A61B 18/20; A61N 5/067

(52) U.S. Cl. 606/4; 607/89

(57) ABSTRACT

A system and method for delivering therapeutic laser energy onto selected treatment locations of the retina following a predetermined spatial distribution pattern using one single laser beam. A beam steering mechanism and control system delivers the laser energy sequentially to treatment locations forming a pre-selected treatment layout pattern. The invention allows time consuming therapeutic laser procedures such as pan-retinal photo-coagulation and segmental photocoagulation to be performed with increased accuracy and in a fraction of the time currently required for such procedures.

### BACKGROUND

1. Field of the Invention

The present invention relates to a modality of laser treatment for the retina, and more particularly, to the delivery of therapeutic doses of laser energy in a rapid sequence to affect a preset pattern of treatment locations during a single burst of laser activity.

2. Description of Prior Art

Laser retinal therapy is used for various ophthalmic conditions requiring therapeutic doses of retinal energy. One example of a multi-dose laser treatment for retinal disease is pan-retinal photocoagulation (PRP). These laser procedures are typically performed using laser delivery systems attached to retinal imaging systems such as a slit-lamp (SL). In most common slit-lamp systems, the laser energy is provided by a laser source to the imaging optics through an optical fiber. The imaging optics are commonly used in conjunction with a variety of contact lenses and are capable of focusing the laser energy exiting the output end of the optical fiber onto the retina, typically in the form of a spot. The focal length of the laser imaging optics is typically variable, i.e. zoom, to magnify the size of the fiber's image on the retina from 1 to 20 times, corresponding to a focused laser beam diameter size of about 50 to 1000 microns on the retinal surface.

Current SL systems offer a single point exposure on the treatment area. The operator positions the laser spot at a desired retinal location by observing an aiming beam on the treatment location. By turning the therapeutic laser ON and then OFF and manually moving the aiming beam during the OFF state, the operator can lay down a plurality of spots on the treatment area. The number of spots is determined by the magnitude of the treatment area and the laser spot size desired. The spot size is selected according to surgeon preferences, pathology to be treated, opacities of the transparent media of the eye and region of the retina requiring treatment, among others. For medical conditions which require PRP, also known as scattered photocoagulation, the area affected may include up to the entire retina outside of the foveal region.

The accepted mode of treatment is to lay down a uniform distribution of photo-coagulation burns, with burn sizes of 100-500 microns and spaced at 1 times the spot diameter. A typical complete PRP treatment consists of between 1000 and 2000 burns. The laser apparatus can be preset to deliver single pulses or a train of laser pulses at fixed intervals during the period a footswitch or other input device is activated by the operator.

Decreasing this interval increases laser pulse repetition rate thus providing a faster treatment. However, there is a practical limit for this repetitive treatment that depends on features of the SL, the type of contact lens being used, patient cooperation and the skill of the operator among others. The time to accurately position the aiming beam onto a new treatment location and the time to deliver the laser photocoagulation burn has to be considered. Aiming beam repositioning time is variable according to retinal visibility, particular area of the retina, magnification and other factors but averages about 0.7 seconds. Typical laser exposure times for obtaining a proper laser burn is in the range of 0.05 to 0.50 seconds. These constrains usually allow no more than 1-1.5 laser spots per second during a typical PRP treatment. This means that the total treatment time can be in excess of 30 minutes, which is fatiguing to the patient and to the surgeon with reduced equipment turnaround time. Also, laying down a uniform pattern of laser burns is difficult and the pattern is typically more random than geometric in distribution.

Multiple laser beams delivery systems have been devised for SL to simultaneously place a plurality of equally spaced laser burns using a single laser pulse source. (U.S. Pat. No. 6,066,128) but have not reached clinical practice.

Also attempts have been made to automate the PRP process by experimenting with complex image analysis software and laser delivery systems but these have not reached the clinical field probably because they are not reliable or cost-effective.

It is a limitation of current slit lamp PRP methodologies the requirement for the operator to reposition the aiming beam onto a new target location each time a laser photocoagulation spot treatment has been placed over a single treatment location.

It is another limitation of current slit lamp PRP methodologies the difficulty in placing an about equally spaced geometrical pattern of laser energy treatment over a plurality of neighbor treatment locations.

It is an overall limitation of current slit lamp PRP methodologies the fact that it is a prolonged repetitive and fatiguing procedure for the operator and the patient.

It is a limitation of multiple beam SL delivery systems the need to multiply the available laser power to simultaneously treat a total larger area of the retina.

It is a limitation of multiple fiber SL systems simultaneously delivering laser power to a plurality of treatment locations the need for higher instantaneous laser powers with potential damage caused to light energy-sensitive ocular structures such as the crystalline lens and others, specially in the presence of opacities.

These high laser energy systems can irradiate the eye structures with energy levels above recommended safety thresholds with the potential of producing light toxicity to the eye of the patient and of the operator. In fact, current SL protective filters could render inadequate to effectively block the more intense light entering the eye of the operator.

It is a limitation of automated image analysis based system proposals for PRP the need of expensive image capturing devices coupled to the SL. It is another limitation of automated image analysis based system proposals for PRP the difficulty in obtaining a simultaneous wide field image of the fundus of the eye that is clear and stable. In this sense, it is an unsolved limitation for these systems the fact that current PRP procedures typically require skillful manipulation of a focusing contact lens and of the SL illumination system to clearly expose, in a sequence, a wide area of the retina for adequate laser treatment. Although confocal non contact systems can be designed for this purpose, the currently used contact lens approach provides the widest field of view and stabilizes the eye to avoid accidental laser delivery onto non-treatable areas.

U.S. Patent Application No. 20060100677 by Blumenkranz et al. included here for reference describes a scanning system for delivering spots of therapeutic laser energy onto the retina substantially in coincidence with spots a pre-positioned alignment pattern. It is an overall limitation of U.S. application No. 20060100677 that this system can only deliver spots of laser treatment in strict coincidence with spots forming a pre-positioned alignment pattern.

Providing an alignment pattern that substantially coincides with the treatment zones that will receive the therapeutic laser energy can over-expose the patient's retina to the operator fixation light and can promote involuntary patient gaze direction toward the treatment area with the potential of causing a laser injury onto the fovea resulting in central blindness.

It is still another limitation of U.S. application No. 20060100677 that this system can only apply spot shaped laser treatments onto the treatment locations. There are circumstances when it is desired that a treatment location has a non-spot shape, i.e. line shaped treatment locations, sometimes placed in parallel to protect the parallel arrangement of nerve fivers traversing at the nerve fiver layer of the retina.

It is still another limitation of the system described in U.S. application No. 20060100677 delivering square shaped patterns of distribution of the treatment locations non optimal by producing uneven distances between neighbor spots, instead of a more physiologic i.e. equidistant pattern, that allows better irrigation distribution and nerve conduction at the remaining retina between neighbor treatment locations.

### OBJECTS AND ADVANTAGES

Among the various objects and features of the present invention may be noted the provision of an apparatus and method which facilitates ophthalmic operations such as pan-retinal or segmental photocoagulation.

Another object is the provision of such apparatus and method which significantly reduces the time required for such procedures being more efficient and well tolerated.

Another object is the provision of such apparatus and method which can be adapted to existing ophthalmic laser treatment equipment.

Another object is the provision of such an apparatus and method which provides increased accuracy and safety for PRP and segmental retinal laser treatments.

Another object is the provision of such an apparatus and method which provides standardized patterns of laser energy to be applied to treatment locations on the retina.

Another object is the provision of such an apparatus and method which sequentially delivers a series of laser treatments in a predetermined spatial pattern onto retinal treatment locations selected using an aiming beam pattern under an operator command.

Another object is the provision of such an apparatus and method where an operator can select the spatial pattern of the treatment locations, width of the treatment locations, separation between the treatment locations, orientation of the pattern of treatment locations and path to be followed between treatment locations during the sequence of individual laser treatments applied to the treatment locations during one burst of laser energy.

Another object is the provision of such an apparatus and method where an aiming beam pattern is positioned by an operator allowing him to clearly identify a retinal area where the laser energy will be delivered to treatment locations.

Another object is the provision of such an apparatus and method where placing of the laser over the treatment locations pattern can be interrupted by the operator at any time

An advantage is the provision of such an apparatus and method where the laser beam power (energy×time) delivered over the retina at any time can vary from treatment location to treatment location according to instrument settings.

Other advantage is the provision of such an apparatus and method where the aiming light pattern delivered over the retina can show the position of the treatment locations using a tracing beam of substantially smaller diameter than that of the laser beam to avoid stimulating the fixation reflex of the patient dangerously directing his gaze toward the treatment area.

Other advantage is the provision of such an apparatus and method where the aiming light pattern delivered over the retina can show the position of the treatment locations using a tracing beam of substantially bigger diameter than that of the laser beam to facilitate visualization of the operator of the treatment locations, for example when opacities of the transparent media of the eye impair good visibility before laser power delivery.

Other advantage is the provision of such an apparatus and method where the laser beam power (energy×time) delivered over the retina at any time can vary from treatment location to treatment location according to instrument settings.

An advantage is the provision of such an apparatus and method where the laser beam power (energy×time) delivered over the retina at any time can remain at the same safe and effective levels currently used to treat known retinal conditions.

Another advantage is the provision of such an apparatus and method where the laser delivery system is cost-effective when compared to therapeutic lasers coupled to image analysis systems.

Briefly, in one aspect of the present invention, a method of performing an ophthalmic surgical procedure such as pan-retinal or segmental photocoagulation on a patient includes the steps of:

(a) directing an aiming beam pattern onto the retina of the patient to select a target area;

(b) transmitting a sequence of laser beams onto the retina over the target area to conform a spatial pattern of laser treatment applied over treatment locations;

(c) directing the aiming beam pattern to a new position on the retina to define an additional target in the retina; and

(d) repeating steps (b) and (c) while necessary.

In another aspect of the present invention, a sequential laser delivery system for performing an ophthalmic surgical procedure such as pan-retinal photocoagulation on a patient includes a source of illumination light, a laser source for generating a beam of laser energy, an optical system for directing the illumination light along an optical path to the eye of a patient to be treated and an optical system for directing the laser energy and the aiming light along an optical path to said eye.

Structure is provided for sequentially steering the beam of laser energy into a plurality of treatment locations to form a predetermined pattern. The steering structure has a distal end through which exit the laser beam following a laser optical path to focus onto the retina. It is preferred that the laser treatment locations have a size suitable for performing the ophthalmic surgical procedure on a human patient.

Further objects and advantages will become apparent from consideration of the drawings and ensuing description.

### BRIEF DESCRIPTION OF THE DRAWINGS

The advantages and features of the present invention will be better understood by the following description when considered in conjunction with the accompanying drawings in which:

FIG. 1A is a first portion of a flow chart illustrating two preferred methods of operation of the present invention.

FIG. 1B is a second portion of a flow chart illustrating two preferred methods of operation of the present invention.

FIG. 2 is a block diagram showing the main components of one embodiment of the present invention.

FIG. 3 is a detailed block diagram illustrating the main interconnections between processor/controller 10 and relevant subsystems and peripherals.

FIG. 4 shows a schematic representation of one embodiment of a laser delivery system of the present invention using a slit lamp.

FIG. 5A shows a top view of a piezoelectric single axis reflective beam steering element used in the preferred embodiment of the present invention.

FIG. 5B shows a side view of a piezoelectric single axis reflective beam steering element used in the preferred embodiment of the present invention.

FIG. 6A shows a top view of an electrostatic single axis reflective beam steering element that can be used in an embodiment of the present invention.

FIG. 6B shows a top view of an electrostatic dual-axis reflective beam steering element that can be used in an embodiment of the present invention.

FIG. 7A shows another schematic representation of an embodiment of a laser delivery system of the present invention suitable for installation in a conventional laser photocoagulation slit lamp.

FIG. 7B is a top schematic view of a low profile dual-axis beam steering unit based on piezoelectric actuators and suitable for use in embodiment 7A replacing mirror 612.

FIG. 7C is a lateral schematic view of the low profile dual-axis beam steering unit shown in FIG. 7B

FIG. 8A shows a detailed lateral view of a low profile piezoelectric dual-axis refractive beam steering system based on relative displacement of spherical lenses of opposing power with the beam in centered position.

FIG. 8B shows a detailed lateral view of a piezoelectric dual-axis refractive beam steering system based on relative displacement of spherical lenses of opposing power actuated to controllably displace the beam off axis at an angle.

FIG. 8C shows a detailed top view of the beam steering mechanism used in the embodiment shown in FIG. 8D with the amplified piezoelectric actuators disposed to displace one lens with respect to another lens of opposing power at parallel planes.

FIG. 9 is an example of selectable options that can be available for an operator at a control panel.

FIG. 10A is a diagram showing a plurality of preset aiming beam patterns selectable by an operator to apply a typical laser treatment pattern based on circularly shaped treatment locations applicable with the present invention.

FIG. 10B is another diagram showing a plurality of preset aiming beam patterns selectable by an operator to apply a laser treatment pattern based on linear shaped treatment locations applicable with the present invention.

FIG. 11 is a block diagram showing alternative embodiments of the present invention related to laser modulation and to aiming overlay data for a retinal imager.

FIG. 12 illustrates one method of the present invention that allows dynamic controller inhibition of laser delivery onto features of the eye fundus using keys derived from a retinal imager.

### LIST OF REFERENCE NUMERALS

Processor/controller 10, user interface 20, eye 50, retina 52, XY beam steering system 60, motion/position controller circuit 62, steering beam therapeutic laser system 100, laser source 102, laser output 103, laser source enclosure 104, aiming light source 106, light sensor 108, beam-splitter/reflector 110, coupling optics 112, light-guide 114, laser delivery system 120, coupler/collimator optics 122, beam profiler 124, beam magnifier 126, controller guided beam steering unit 128, primary surface mirror 130, beam splitter 131, retinal focusing optics 132, retinal imaging contact lens 134, aiming steering mechanism 136, beam-splitter/reflector 138, retinal imager 140, beam position sensor 142, light filter/blocker 144, laser inhibit input 200, laser power input, laser wavelength input 204, microprocessor 300, memory 310, external trigger 320, joystick 340, refractive dual axis beam steering unit 550, incident beam axis 552, emerging beam axis 556, fixed frame 558, lens holder 560, fixed refractive element 570, movable refractive element 572, steering angle 576, first piezoelectric actuator 580, actuator power signal 582, actuator position sensor signal 584, second piezoelectric actuator 590, second actuator power signal 592, second actuator position sensor signal 594, retinal illuminator 600, retinal illumination light 602, steering beam 604, aiming beam 606, laser beam 608, filter/blocker 610, mirror 612, imager objective 614, imager eyepiece 616, operator eye 618, single axis piezoelectric beam steering unit 700, primary surface mirror 702, mirror pivoting axis 704, actuator-mirror coupler 706, piezoelectric actuator 708, base plate 710, position sensor 712, actuator power signal 714, single axis electrostatic beam steering unit 720, primary surface mirror 722, mirror pivot axis 724, dual axis electrostatic beam steering unit 730, primary surface mirror 732, mirror pivot on X axis 734, mirror pivot on Y axis 736, movable frame 738, digital mirror device 800, condenser 802, light absorber 804, active deflection angle 810, 850, aiming display 850, beam splitter 852, image sensor 860, compact dual axis beam steering unit 900, primary surface mirror 902, piezoelectric actuator mirror couplings 906, base plate 910.

### SUMMARY

In accordance with the present invention it is an object to provide an optimized laser treatment system to deliver laser energy sequentially onto pattern of treatment locations of a patient retina using a rapid laser beam steering mechanism. An aiming system allows an operator using a retinal imaging system to select a treatment area where the pattern of retinal treatment locations will sequentially receive doses of therapeutic laser energy. Each burst of laser energy is typically completed in less than 1000 milliseconds. The operator can then select a new treatment area using the aiming system. A laser shutter mechanism can be incorporated to avoid exposure of the retina located between treatment locations to undesired levels of laser energy while the steering mechanism is moving the laser beam between one treatment location and another treatment location.

### DETAILED DESCRIPTION

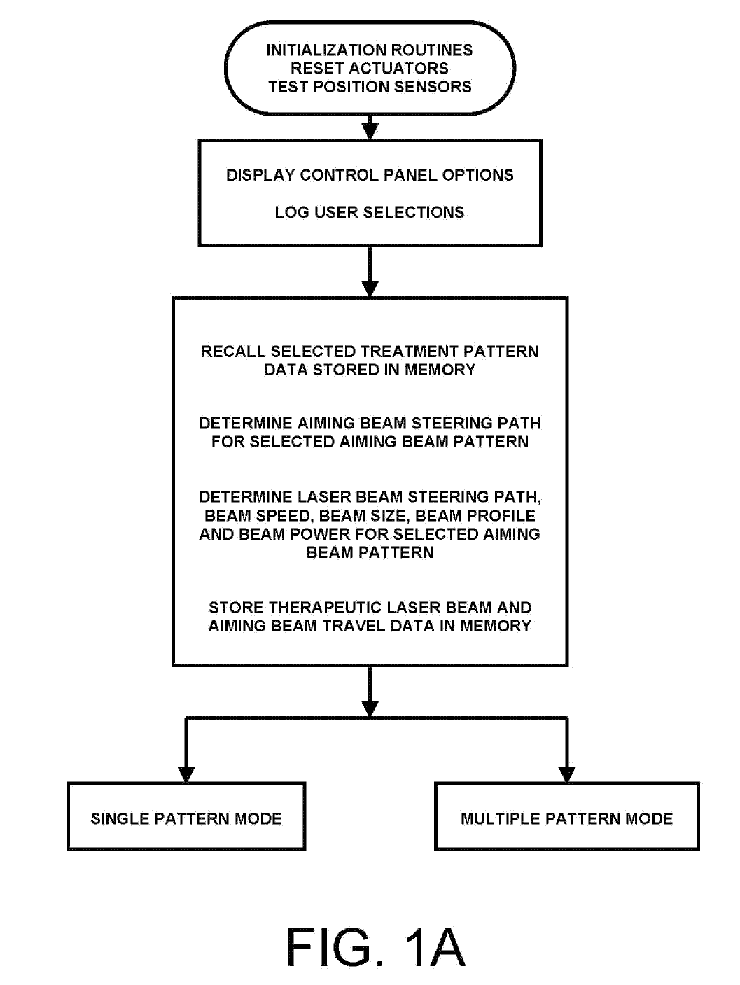

FIGS. 1A and 1B depict a flow diagram a method that can be practiced with the present invention. Generally speaking, the steps of this method consider setting the system for operation including making selections related to the planned treatment, having a processor/controller 10 process and store relevant data according to the operator selections, projecting an aiming beam pattern onto retinal areas, using said aiming beam pattern as a reference, selecting a treatment area of the retina where therapeutic doses of laser energy will be delivered over treatment locations disposed in the predetermined treatment pattern. The operator performs a triggering action having the system to deliver a rapid burst of modulated laser energy while rapidly steering the laser beam to obtain the desired pattern of treated locations on the retina.

The operator can adjust aspects of the selected pattern of treatment locations between applications, including repositioning, rotation, scaling, and sizing eventually using an aiming light projected over the retina as a reference for performing these tasks.

Typically, the delivery of therapeutic laser energy is initiated by an operator command such as depressing a foot switch, a button, or any other input device to controllably produce a trigger signal. When activated, the system delivers a plurality of doses of therapeutic laser energy in a rapid sequence using at least one steering laser beam. The doses of therapeutic laser energy are delivered following a spatial and temporal pattern over pre-selected treatment locations of the retina. Preferably all the doses of therapeutic laser energy are delivered during a burst of laser activity lasting less than 1000 milliseconds. Anyway, longer lasting therapeutic laser energy bursts can be programmed according to particular conditions of a procedure.

The therapeutic doses of laser energy can be delivered in sequence to multiple treatment locations of the retina oriented by an aiming beam pattern. The therapeutic laser pattern and the aiming beam pattern are not required to be spatially similar to practice this invention.

In an alternative embodiment the aiming beam pattern is replaced by an aiming pattern produced by a display unit 850 under processor control and overlaid onto the retinal image produced by a retinal imager 140. In this embodiment, other treatment related information can be included in the overlay image to help an operator complete a treatment session. Regardless of whether the aiming process is performed using an aiming beam pattern directly over the retina or a processor generated overlay pattern for a retinal imager, each operator triggering action initiates a new burst of laser activity to create a therapeutic laser pattern onto a new selected area of the retina.

The system and methods to practice this invention provide reduced treatment time for multi-dose photocoagulation procedures. By delivering all the doses of laser energy in a time less than an eye fixation time, the requirement for expensive retinal tracking systems is eliminated, since the eye can be expected to remain steady during each laser treatment burst to complete the treatment pattern. When necessary, steadiness of the eye during treatment can by enhanced by holding a contact lens over the anterior portion of the eye during system operation.

Several methods can be used to repeatedly deliver patterned therapeutic doses of laser energy onto treatment locations that compose a treatment pattern. For example, FIG. 1A and FIG. 1B describe a multi-burst method where a timer is set after delivery of one treatment pattern. In this modality, if the operator sustains the triggering action, once the timer period is completed, a new burst of therapeutic laser energy is automatically triggered to produce a new pattern over treatment areas. It is expected that the operator has proper time to reposition the aiming beam pattern during this lapse to select a new area or retina to receive a treatment pattern. This repetitive burst action can further enhance efficiency of this therapeutic laser system.

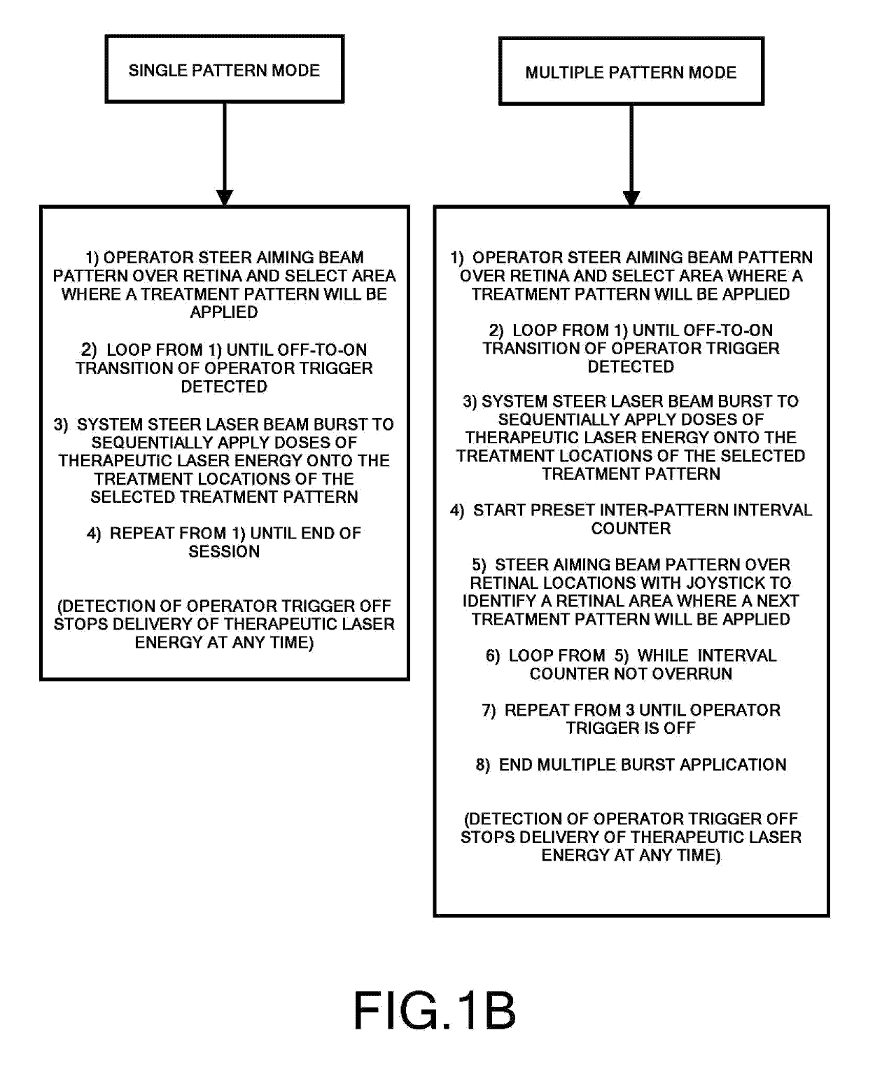

FIG. 2 depicts a schematic diagram of a pattern photocoagulation system 100 of the present invention suitable for performing the methods described in FIG. 1A and FIG. 1B. FIG. 2 also depicts an eye 50 with a retina 52 where therapeutic doses of laser energy can be applied onto treatment locations. System 100 includes a processor/controller 10, a user interface 20, a laser source 102 and a laser delivery system 120 all suitably interconnected. Laser energy is delivered by laser source 102 at a laser output terminal 103.

Processor/controller 10 is capable of providing computation power at a speed suitable to simultaneously control in real time the operation of laser source 102 and of laser delivery system 120 including all its components to precisely deliver in sequence a beam of therapeutic laser energy onto a pattern of desired locations of retina 52 with the required speed.

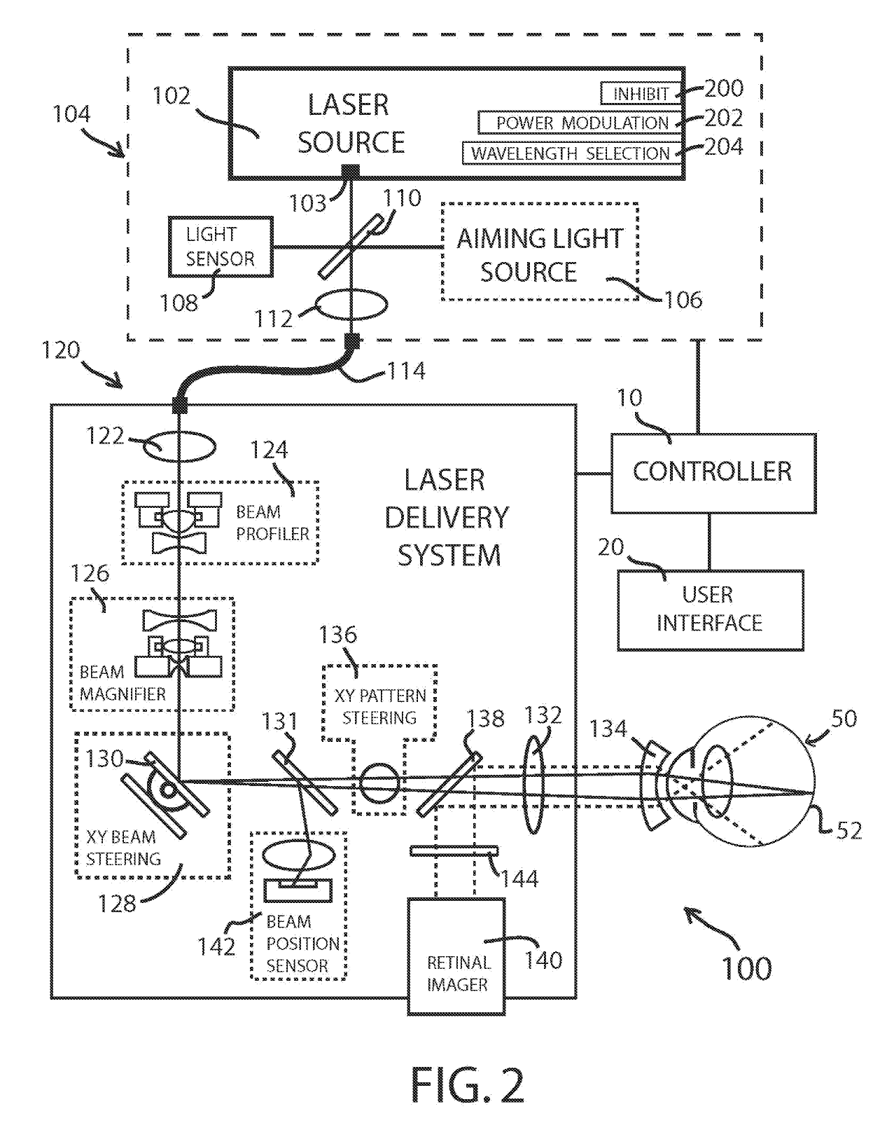

Specifically processor/controller 10 should include at least one microprocessor 300 as shown in FIG. 3. Processor/controller 10 is connected by suitable data conductors to the sensors and actuators required for operation of system 100. User interface 20 is also interconnected with processor/controller 10. User interface 20 provides input signals such as operator selections at a control panel and triggering actions on a trigger 320 as well as output signals such as visual information including treatment relevant data and audio signals related to treatment progression.

Laser source 120 can be enclosed within a laser source compartment 104 separated from laser delivery system 120. Laser energy is conducted between laser source 102 and laser delivery system 120 using a suitable light guide 114 such as fiber optic conductors. Laser source 102 can also be incorporated inside laser delivery system 120. Laser source 120 is selected to deliver therapeutic doses of laser radiation, typically with a maximum RMS power above 2.500 watts.

The wavelength of the laser radiation produced by laser source 102 is typically in the visible or infrared portions of the electromagnetic spectrum to produce the therapeutic effect, although other wavelengths could be required for future treatments. Typical laser emission wavelengths selected for retinal treatment are 512 nm and 810 nm. In the case of a wavelength of 810 nm, the therapeutic laser energy is invisible to the operator's eye.

Laser emission by laser source 102 can be selected to be continuous or pulsated at high speed, typically at frequencies above 20 kHz according to specific treatment objectives. Laser source 120 can provide a laser inhibit input 200 to receive a laser inhibit command from processor/controller 10 in the way of an electric signal, optical signal, radio signal or any other suitable means for fast data transmission.

Inhibit input 200 is capable of shutting down laser energy output at laser output 103 with a response time of less than 0.05 milliseconds. After termination of an inhibit signal applied to laser inhibit input 200, laser energy output must be turned ON at laser output 103 to the preset output level with a response time of less than 0.05 milliseconds. This laser inhibit feature can allow control of the laser output by processor/controller 10 to produce a burst of switched laser activity during patterned therapeutic laser delivery.

Laser source 120 can also provide a laser power modulation input 202 to receive power modulation commands from controller/processor 10. Laser source 102 adjusts the output power available at laser output 103 between OFF and a MAXIMUM output power following controller/processor 10 commands. Power modulation algorithms to regulate the output power of laser source 102 can consider amplitude modulation, pulse width modulation or any other power modulation schemes suitable for adjusting laser 102 power output. This power modulation feature allows control of laser output by processor/controller 10 during a burst of therapeutic laser activity.

Laser source 120 can provide a laser wavelength selection input 204 to receive wavelength selection commands from processor/controller 10 to adjust the wavelength of the laser energy at laser output 103 according to treatment preferences. With this feature processor/controller 10 can select a visible wavelength to produce a visible laser beam pattern for the aiming process and then select an invisible wavelength for the treatment pattern.

Laser compartment 104 can enclose an optional aiming light source 106 and a light sensor 108. A beam-splitter/reflector element 110 suitably disposed in the light path optically connects laser output 103, aiming light source 106, light sensor 108 and coupling optics 112. Aiming light source 106 is preferably a low power visible light source provided by a LED or a solid state LASER adjusted to provide safe levels of light radiation to retina 52 at any time during the duration of a treatment session. Aiming light source 106 can be replaced by visible light produced by laser source 102. In this situation processor/controller 10 adjusts the output power of source 102 for aiming purposes providing light levels compatible with safe standards of aiming light at retina 52. The power adjustment process by processor/controller 10 can consider but is not restricted to electronic power modulation using inputs 200 and 202, partial deflection of output laser energy at laser output 103 using a digital mirror device 800, mechanical insertion of attenuation filter, etc. Light sensor 108 provides information about aiming light and therapeutic laser light levels to processor/controller 10 for monitoring purposes.

Coupling optics 112 are disposed to receive light energy available from laser output 103 and by optional aiming light source 106 and to transmit this light energy to a light guide 114 such as a multimodal optical fiber selected to optimally transmit the wavelengths of the lights emitted by light sources 102 and 106. Laser delivery system 120 receives light-guide 114 through coupler/collimator optics 122.

A beam profiler 124 can be suitable disposed along the light path between coupler/collimator 122 and a pattern beam steering mechanism 128. Beam profiler 124 is designed to modify the laser beam profile in response to commands transmitted by processor/controller 10, and for example, has the capability of changing the laser energy distribution pattern among a selection of laser beam patterns such as Gaussian, inverse Gaussian, hat top or any other suitable form of laser beam profiling. Beam profiler 124 can be based on an electro-optical assembly driven for example by motors or piezoelectric actuators.

A beam magnifier 126 can be disposed along the light path between coupler/collimator 122 and beam steering mechanism 128. Beam magnifier 126 is designed to modify the laser beam diameter typically by a factor ranging between 1 and 20 in response to commands applied by processor/controller 10. Alternatively beam magnifier 126 can be manually set by an operator in which case the beam diameter selection can be provided as an input to processor/controller 10 for accurate laser beam pattern data calculations. Beam magnifier 126 can be based on an electro-optical assembly driven for example by motors or piezoelectric actuators.

The light path output from coupler/collimator 122 is received by beam steering mechanism 128. Beam steering mechanism 128 is selected to provide dual axis steering capabilities and a high speed of operation. Step settling times below 2 milliseconds for steps above 400 microns at the retinal plane are preferred for practicing this invention to its full potential. Beam steering mechanism 128 must provide a minimum steering range of 500 microns diameter at the retinal plane. This minimum steering range allows for treatment patterns limited to 3 to 9 treatment locations of 200 microns. It is desirable that the steering range of beam steering mechanism 128 covers a diameter of 4 millimeters at the retinal plane to be capable of producing treatment patterns including 10 equally spaced 200 micron sized treatment locations disposed along any diameter of the steering area.

In the embodiment shown in FIG. 2, laser delivery system 120 incorporates a reflective beam steering mechanism having a primary surface mirror 130. Beam steering mechanism 128 is under control of processor/controller 10 to steer the laser beam along the steering area to apply doses of therapeutic laser energy to the retina in the predetermined pattern configuration with a speed and accuracy much higher than that possible for a human operator.

The light path exiting beam steering mechanism 128 is directed across beam focusing optics 132 to focus the steering light beam onto a focusing plane at retina 52. An optional lens 134 can be placed along the light path exiting the laser delivery system 120 and the anterior portion of the eye 50. Lens 134 can be any suitable retinal imaging lens such as a contact lens, an inverting lens, a mirrored lens with the purposes of improving retinal imaging of the treatment area, scaling the retinal image, scaling the treatment location sizes and/or holding eye 50 steady during treatment.

A beam splitter element 131 can provide a light path for partial reflection of the steered light beam into a beam position sensor 142. Beam position sensor provides real time information of operation of the pattern beam mechanism 128 to processor/controller 10 for servo-control and for system monitoring purposes. A beam position sensor detector 142 suitable for performing this task at the required speed and precision is the Duo-Lateral Super Linear PSD DL-10, from OSI Optoelectronics, USA, although other sensors are also suitable for this task.

A human operable pattern steering mechanism 136 typically independent of beam steering mechanism 128 is disposed to steer the light beam in a relatively wide field of retina 52, preferably above 4 millimeters diameter to allow an operator to use an aiming light produced by laser source 102 or by aiming light source 106 to select new areas of the retina suitable to receive a next patterned application of therapeutic laser energy.

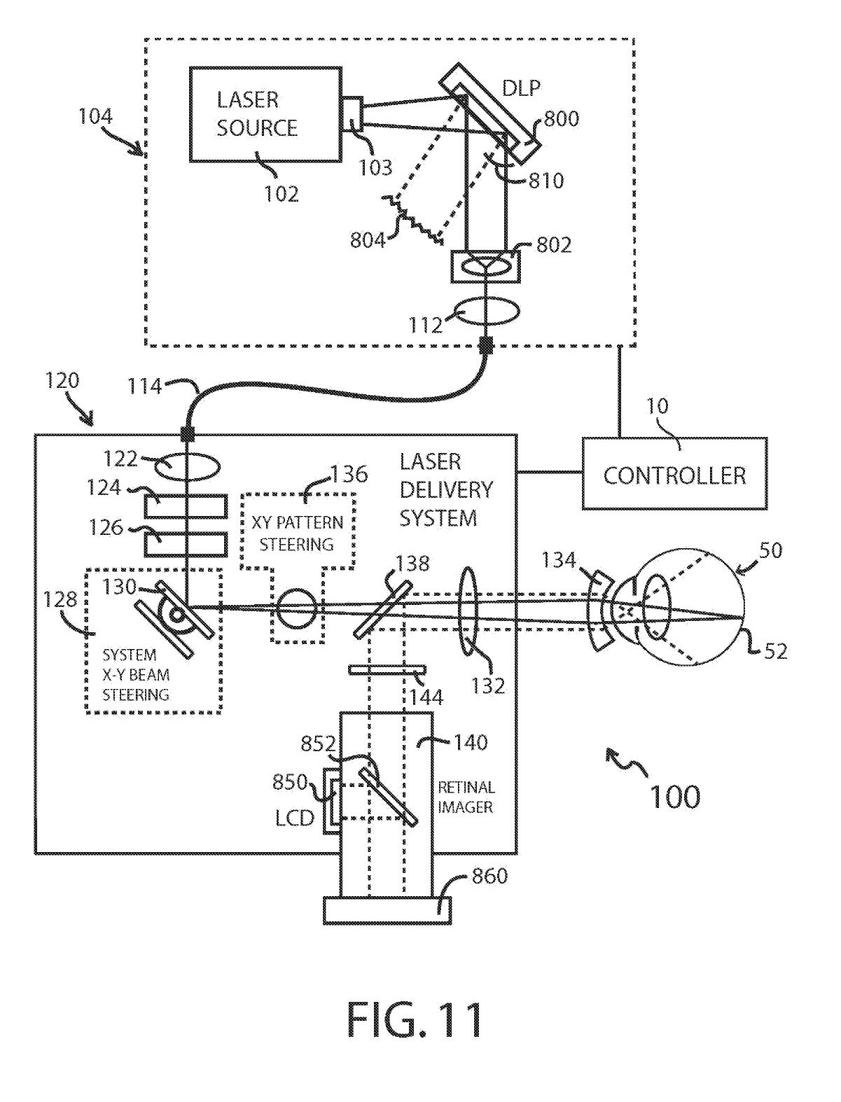

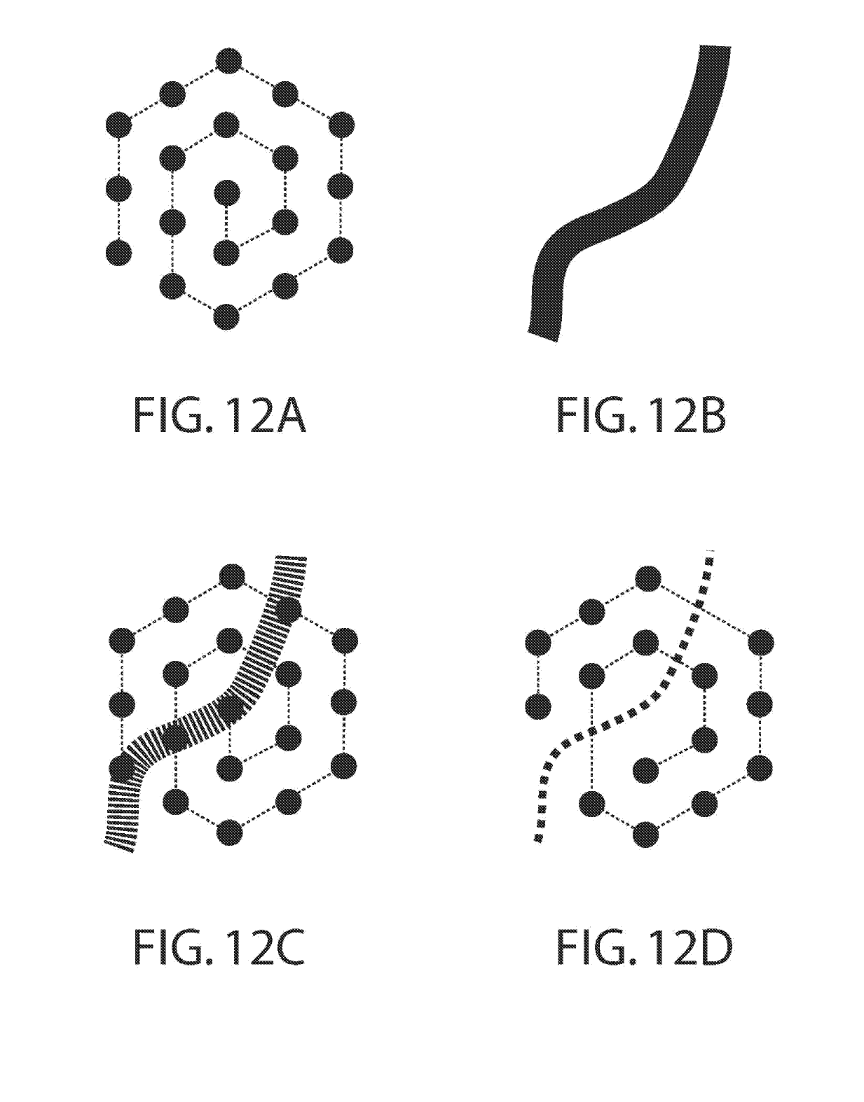

Typically, an XY input device such as a joystick 340 is used by an operator to command pattern steering mechanism 136 to proportionally displace along retina 52 with the aiming light to select new locations for patterned treatment. Other pointing devices such as a computer mouse, trackball, etc, can be equally suitable for the task of selecting treatment pattern locations on a patient retina 52 particularly when indirect retinal imaging systems are employed as described in FIG. 11.

A beam-splitter element 138 can be disposed in the light path between pattern steering mechanism 136 and retina 52 to allow a retinal imager 140 to focus an image of retina 52 for aiming and treatment monitoring purposes. A shutter/filter 144 can be disposed along the light path between beam-splitter 138 and retinal imager 140. Shutter/filter 142 is suitably selected and operated to protect the receiving optics including a human eye from noxious radiations derived from laser delivery system 120 and/or reflected by eye 50. Shutter/filter 142 can be a notch filter that selectively blocks the wavelength of the therapeutic laser energy. Shutter/filter 142 can also be a mechanical device, an LCD based attenuator or other suitable laser energy protecting element.

Turning now to FIG. 3, interconnections between processor/controller 10 and main peripheral units are detailed. Microprocessor/controller includes a microprocessor 300 such as a dsPIC30F Digital Signal Controller form Microchip Corporation, USA, capable of performing at least 20 MIPS. Other options of microprocessors can be considered, preferring high speed, control oriented digital signal processors capable of accurate time computations and real time motion control processing. Processor/controller 10 can be conformed by a plurality of microcontrollers, sensor and control subsystems to enhance performance.

Microprocessor 300 includes a memory module 310 divided into non volatile and volatile memory to store programs and operational data. Optionally, EEPROM memory or other storage systems can be used to store user preferences and patient data. Microprocessor 300 can be connected to laser source 102 to provide an inhibit signal through input 200, a power modulation signal through input 202 and a wavelength selection signal through input 204. Microprocessor 300 is suitably interconnected with user interface 20 where a control panel can include a touch screen, graphic display, keyboard, etc, for operator selections, procedure programming and treatment related feedback data.

A triggering input device 320 such as a footswitch is incorporated to user interface 20 connecting with microcontroller 300. A joystick 340 providing an XY information signal to steer an aiming beam along the retina using operator steering mechanism 136 is connected to an input of microprocessor 300 for aiming and treatment purposes. The output signal of laser beam position sensor 142 is connected to microprocessor 300 to provide real time information of the XY position of the laser beam for feedback and servo control purposes.

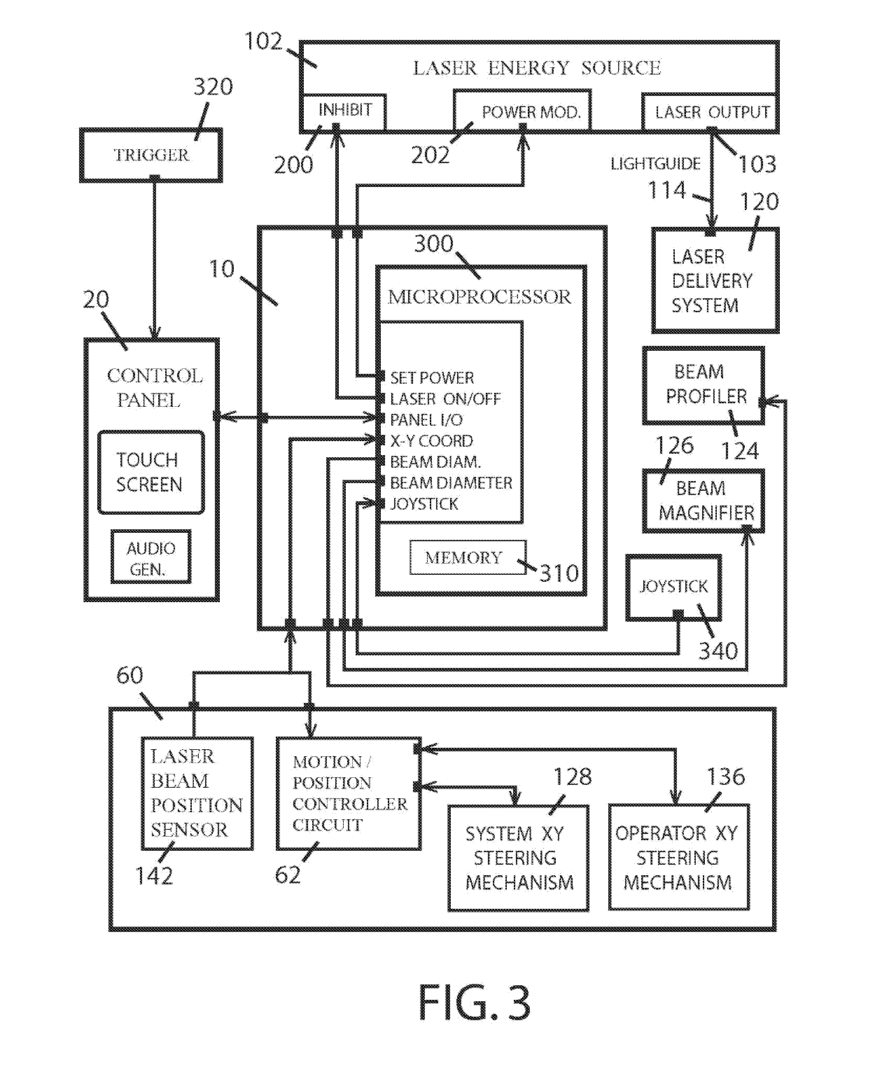

Microprocessor 300 provides control signals for a motion/position controller circuit 62, which provides the power signals for the actuators involved in system related beam steering mechanism 128 and in operator related pattern steering mechanism 136. Microprocessor 300 can provide a control signal for beam profiler 124 and for beam magnifier 126 for adjustments of the beam profile and beam size. In situations where an electro-mechanic dynamic beam magnifier 126 is not included, it is an option to have an input signal into microcontroller 300 coming from a manually set beam magnifier 126 of FIG. 4 informing the processor of the selected beam diameter for laser beam pattern computations.

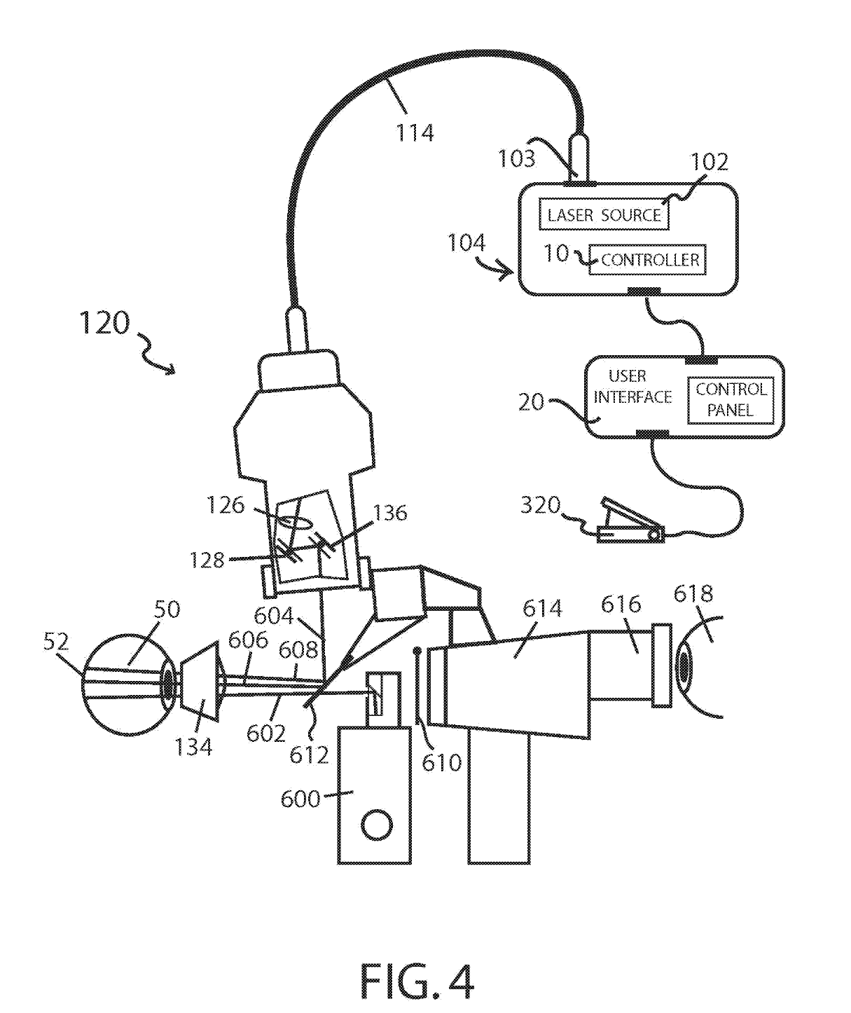

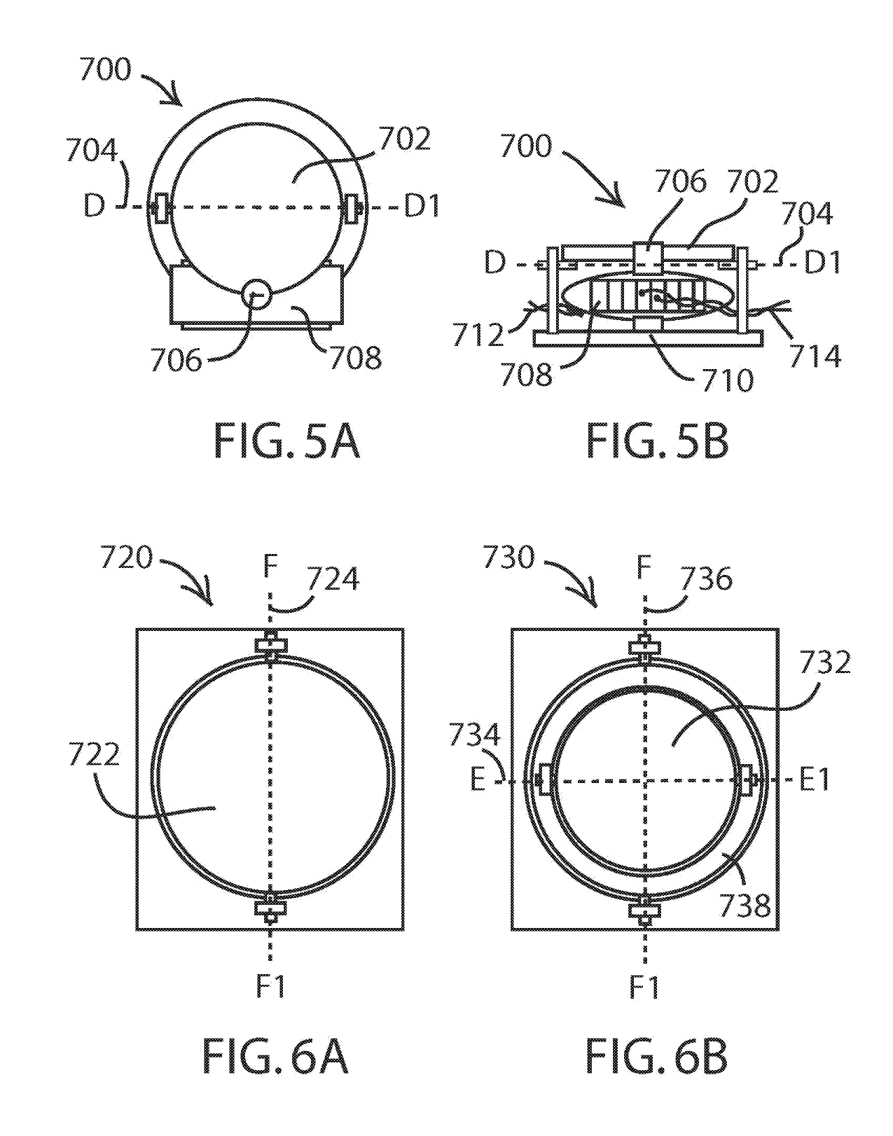

In FIG. 4, a schematic view of one embodiment of the present invention based on a slit lamp for retinal imaging purposes is presented. A fiber-optic light guide 114 carries laser and aiming light power into laser delivery system 120 including an adjustable laser beam magnifier 126. Beam steering unit 128 enclosed within laser delivery system 120 can be composed of two single axis or one twin axis beam steering unit such as shown in FIGS. 5A, 5B, 6A and 6B disposed to produce fast and accurate XY laser beam displacement under processor/controller 10 command. The slit-lamp based system includes a retinal illuminator 600 capable of diffusely illuminating a relatively wide portion of retina 52 of eye 50 following light path 602. An operator can look into an eyepiece 616 with his eye 618 to view an image of retina 52 across an objective 614 illuminated by light beam 602.

Steering light beam 604 has a dual purpose. During the aiming process it carries aiming light that composes an aiming pattern. During the treatment process it carries bursts of therapeutic laser light steered to conform a treatment pattern. Processor/controller 10 has the task of properly driving beam steering unit 128 and providing the proper levels of light for both the aiming and the treatment processes. Beam 604 is directed to a mirror 612. Mirror 612 reflects aiming light preferably towards an illuminated portion of retina 52 across a light beam 606. Also, mirror 612 reflects therapeutic laser light emerging from steering mechanism 128 across laser beam 608 towards a portion of retina 52 selected to receive patterned doses of therapeutic laser energy using aiming light 606 for reference. Contact lens 134 can improve visualization of the treatment portion of retina 52 and can enhance eye 50 stability during a procedure. A laser filter/blocker 610 is disposed to protect an eye 618 of an operator from dangerous light energy.

Pattern steering mechanism 136 can be a conventional slit lamp laser beam steering system, i.e. manual or motor powered, and can be located inside laser delivery system 120 in a way to provide wide field movements of the aiming pattern over retina 52. Operator commanded wide field XY pattern steering mechanism 136 must be capable of operating at a speed matching the speed a human operator can operate a joystick 340 or similar aiming device. Operation of joystick 340 activates the aiming steering mechanism with proportional control. It can be convenient to have processor/controller 10 electrically block joystick operation while a burst of therapeutic laser power is being applied to the retina for increased accuracy. Electronic processing of the operator provided command signal for operator steering system 136 can help adjust the response time and apply filters to avoid jerky or unsafe operation of the aiming beam and treatment beam delivery systems.

FIG. 5A shows a top view of a single axis reflective piezoelectric actuated beam steering unit 700. A primary surface mirror 702 has a pivot axis 704 across a line drawn between D and D1. Turning to the lateral view of FIG. 5B it can be appreciated that an amplified piezoelectric actuator 708 is disposed to tilt mirror 702 along pivoting axis 704 when expanding and contracting between base plate 710 and mirror coupling 706. An actuator power signal 714 is received from motion/position controller circuit 62 and an actuator position sensor signal 712 is also sent controller circuit 62 under processor/controller 10 supervision. Each beam steering unit 700 receives independent power signals 714 and delivers individual position sensor signals 712.

An alternative to piezoelectric actuated steering mechanisms is the use of electrostatic actuated steering mechanisms. FIG. 6A depicts a single axis electrostatic beam steering element 720 including a mirror 722 pivoting around an axis 724 traced with a line between F and F1. FIG. 6B depicts a dual-axis electrostatic beam steering element 730 including a mirror 732 pivoting around an axis 734 traced with a line between E and E1. A movable frame 738 can pivot around an axis 736 traced with a line between F and F1 displacing mirror 732 in a perpendicular axis with respect to the element base plate. An example of an electrostatic dual axis steering mirror suitable for implementation with this invention is the Scanning Two Axis Tilt Mirror Device from MemsOptical Inc, USA.

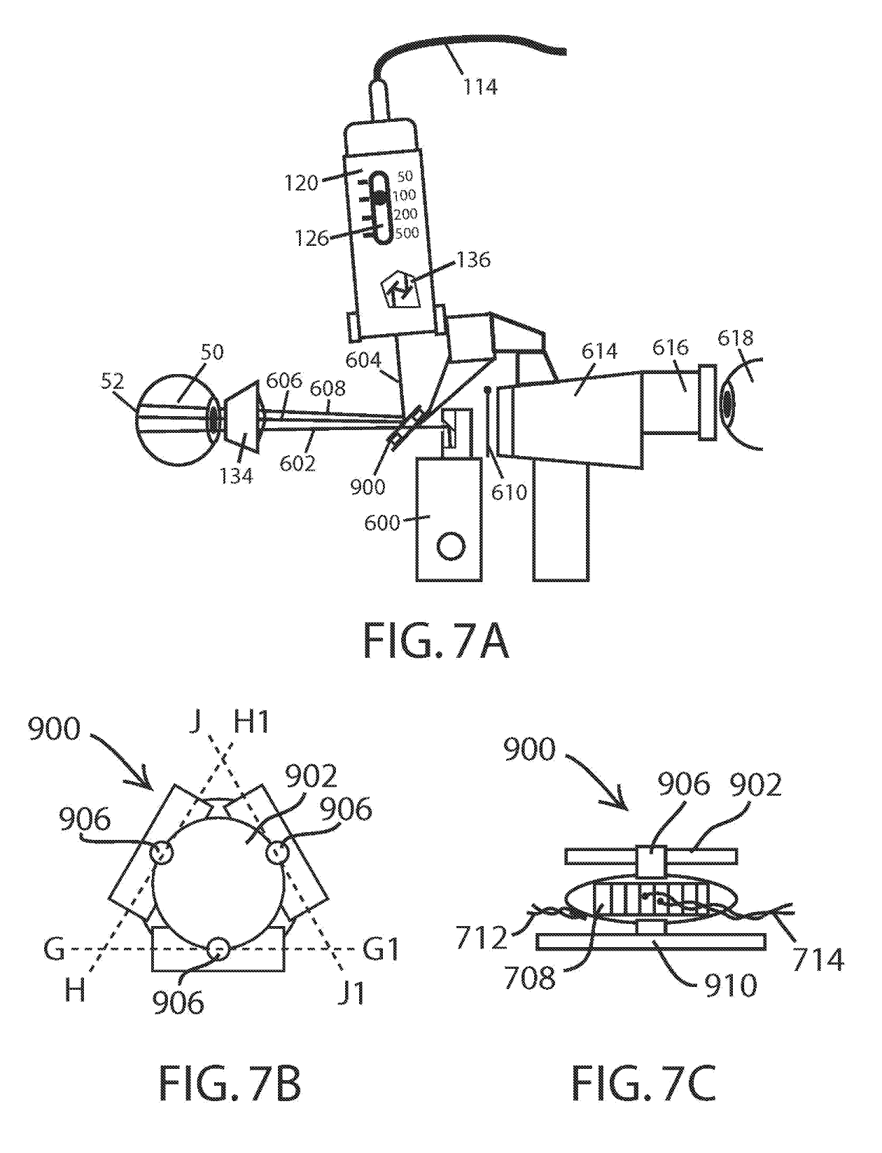

FIG. 7A illustrates an alternative embodiment of the steering laser system of the present invention suitable for implementation into conventional photo-coagulation slit lamps. Mirror 612 shown in FIG. 4 is replaced by a high speed, compact XY beam steering unit 900. This beam steering unit is better described in FIG. 7B and FIG. 7C. A mirror 902 is coupled with couplings 906 in a triangular array onto the free ends of three amplified piezoelectric actuators with a displacement of 80 microns each between base plate 910 and mirror coupling 906. Each beam steering unit 900 receives independent power signals 714 and delivers individual position sensor signals 712 to motion/position controller circuit 62. An electrostatic operated Scanning Two Axis Tilt Mirror Device can be used instead of a piezoelectric beam steering unit in the location of actuator mirror 900.

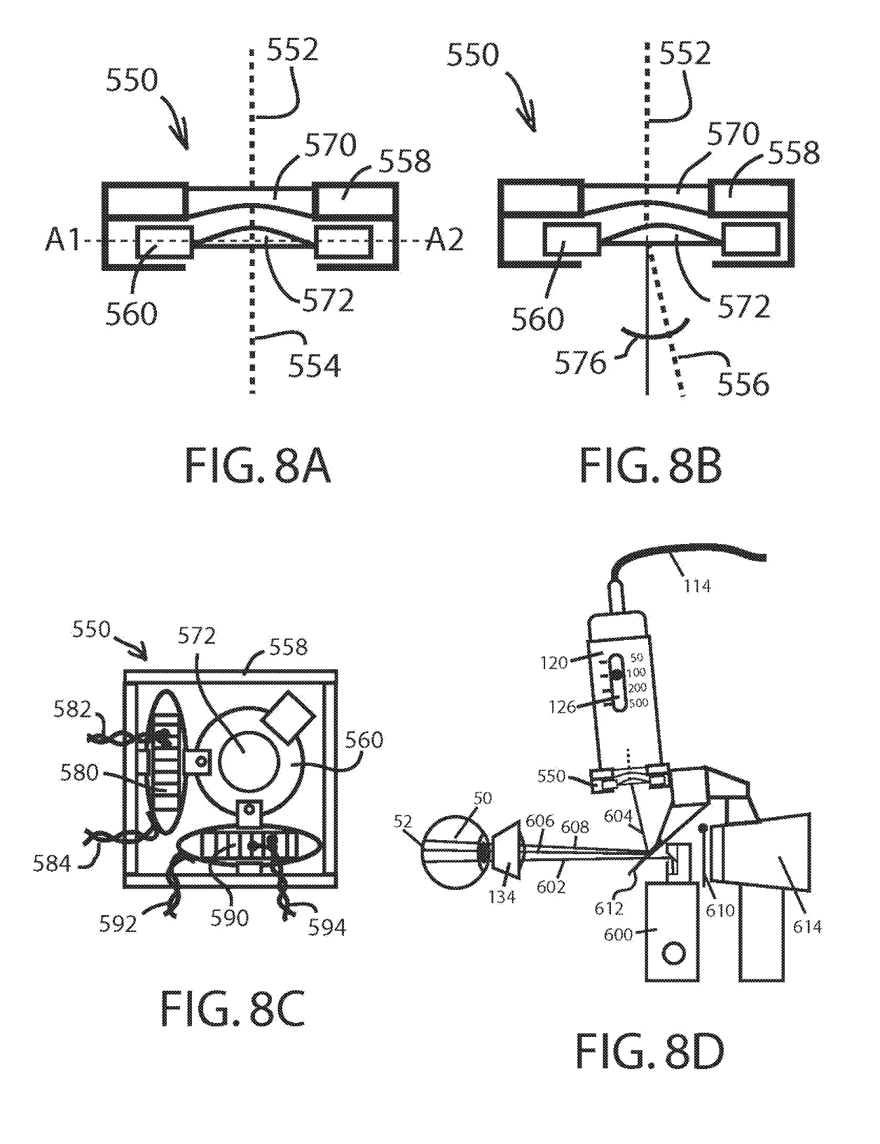

FIGS. 8A and 8B illustrate an alternative embodiment of the steering laser system of the present invention suitable for implementation into conventional photo-coagulation slit lamps. A refractive dual-axis beam steering mechanism 550 is depicted. A frame 558 holds a pair of spherical or aspherical lenses of opposing dioptric power in close proximity. In the depicted embodiment a lens 570 is fixed to frame 558 and has negative power. Parallel to lens 570 and on the same axis a lens 572 of opposing positive dioptric power is mounted on a movable lens holder 560 firmly coupled to two perpendicularly disposed piezoelectric actuators as shown in FIG. 8C. Combined operation of piezoelectric actuators 580 and 590 allows relative XY displacement of lens 572 relative to lens 570 in all directions. When both lenses coincide in their optical axis, a narrow perpendicular beam traverses the unit in a straight line. Activation of piezoelectric actuators 590 and 580 producing an offset of the optical axis of both lenses produces an axis shift toward the displacing direction of lens 572 producing a steering angle 576 proportional to axis offset. This effect is explained by a principle related to the optics of prisms. FIG. 8D illustrates the refractive dual-axis beam steering mechanism 550 of this embodiment installed at the laser delivery port of a conventional photo-coagulator system. This beam steering mechanism can be used to perform the functions of beam steering units 128 and/or 136.

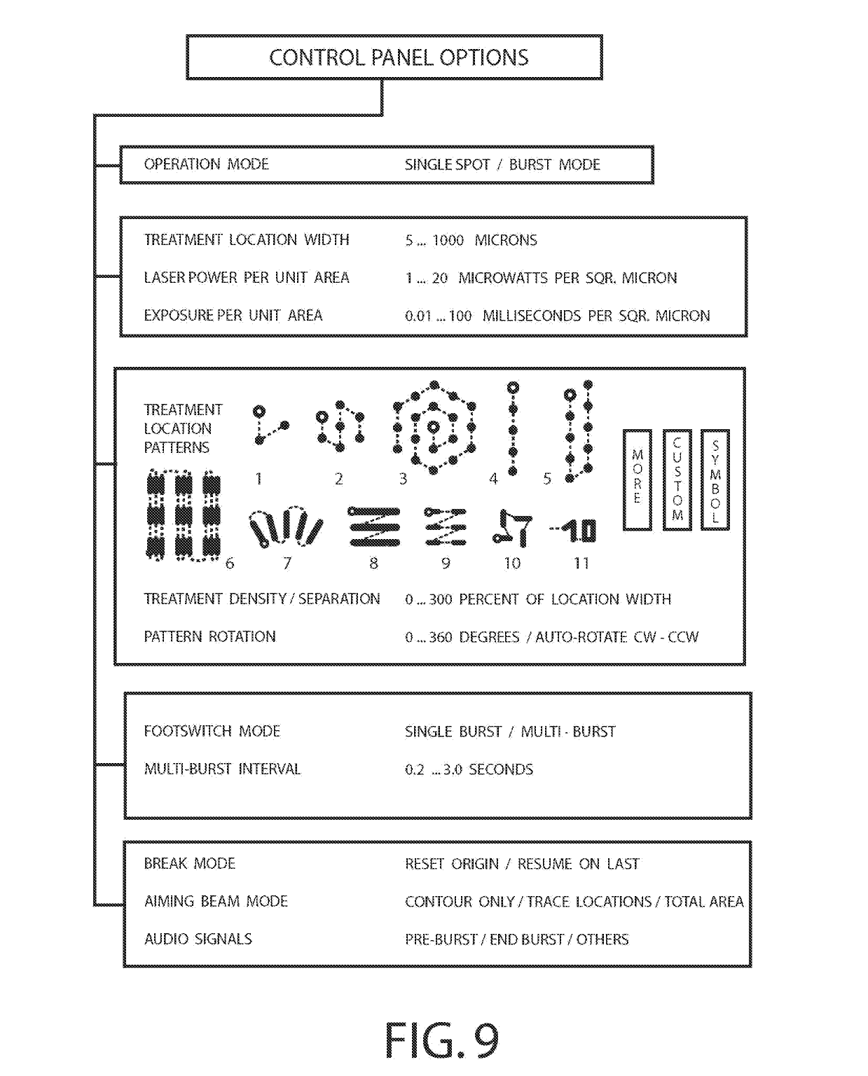

FIG. 9 illustrates examples of control panel options that can be available for an operator at user interface 20 including a series of user selectable therapeutic laser treatment patterns. These treatment patterns include steady beam and moving beam treatment pattern modalities. Processor/controller 10 has access to memory data relative to each treatment pattern, including steering beam path vectors, speed of travel and power modulation data.

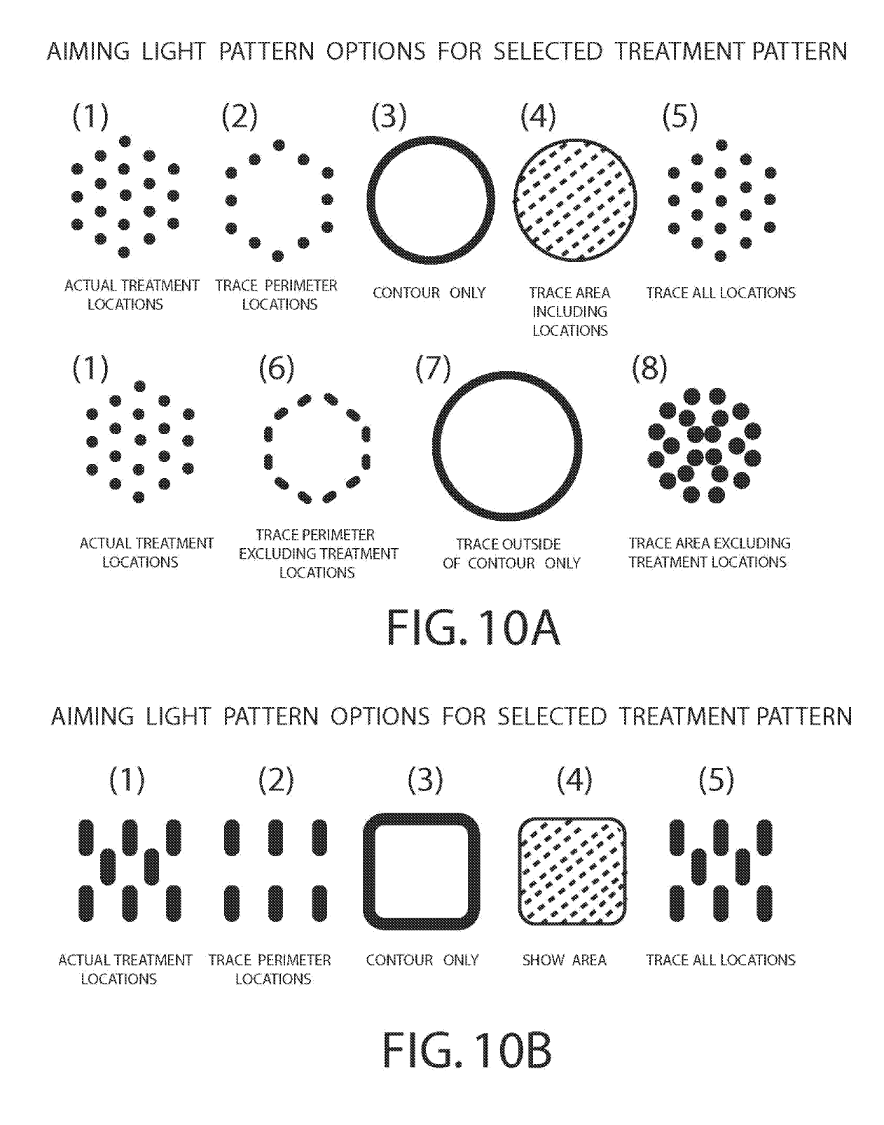

FIG. 10A illustrates suggested aiming light pattern options selectable by an operator to help position treatment patterns in selected areas of retina 52. An hexagonal pattern of 19 equally spaced triangularly disposed treatment locations FIG. 10A(1) has been selected for example. To help positioning this particular treatment pattern over a selected area of the patient retina, an operator can select among a plurality of aiming light options to select the treatment areas over an illuminated portion of retina 52 using joystick 340.

As a mode of example, the tracing light can illuminate the most peripheral treatment locations of the selected treatment pattern FIG. 10A(2). The tracing light can draw a continuous line delimiting the contour where the selected treatment pattern will fall within FIG. 10A(3). The tracing light can illuminate the complete area where the treatment pattern will fall FIG. 10A(4). The tracing light can illuminate locations that will match with the treatment locations of the treatment pattern FIG. 10A(5). The aiming light can trace a perimeter of the treatment pattern area excluding the area of the actual treatment locations that will fall in said perimeter FIG. 10A(6). The aiming light can trace a perimeter that concentrically delimits an area that is an amount bigger that the actual perimeter of the treatment pattern that will be laid on the selected treatment area FIG. 10A(7). Also, the aiming light can draw a pattern that illuminates the treatment area excluding the treatment locations of the selected pattern FIG. 10A(8). It can be understood that these aiming beam selection patterns are exemplary only, and that many other aiming light patterns can be used without departing from the scope of the present invention.

FIG. 10B depicts some examples of aiming beam patterns for use with the present invention. In this case the treatment pattern is composed of 8 line shaped parallel treatment locations. The same aiming options illustrated in FIG. 10A are here illustrated with the numbers FIG. 10B(2) to FIG. 10B(5) for this different treatment pattern.

The schematic diagram of FIG. 11 shows an alternative power modulation scheme for laser source 102 of laser system 100 of the present invention. A laser source 102 provides a collimated beam of therapeutic laser energy at laser output 103 aimed in a specific incident angle onto the reflective surface of a digital mirror device 800. Mirror device 800 can be a micro-mirror Digital Light. Perception (DLP) device, such as 0.55 VGA DLP from Texas Instruments, USA, consisting of 307.200 active micro-mirrors in an array of 640×480 elements, each mirror being capable of tilting an angle 810 of 20 degrees at a frequency above 100 kHz (0.01 milliseconds period) in response to a digital signal. Each mirror element can be individually operated by the controlling processor.

The beam of laser energy reflected by each micro-mirror element of digital mirror device 800 can be directed toward a light absorber 804 or toward a condenser 802 according to the logic status of the controlling bit for that particular mirror element upon processor/controller 10 command. Condenser 802 can collect the laser light reflected by the active (ON) mirrors of device 800 while absorber 804 receives the light reflected by inactive (OFF) mirrors of device 800. Laser light absorber 804 can incorporate a condenser and a laser power sensor 108 connected to processor/controller 10 to monitor the output power of laser energy source 102 and simultaneously monitor operation of active mirror device 800. Collimator optics 112 couple the output light from condenser 802 onto light-guide 114. In cases where the therapeutic laser energy laser source 102 produces non-visible light such as infrared, an aiming light source 106 can be directed to a dedicated portion of device 800.

Also shown in FIG. 11 is an alternative embodiment for an aiming system for the therapeutic laser system 100 of the present invention. Beam splitter 138 located in the exit light path of steering unit 128 provides light from retina 52 for retinal imager 140 where an image sensor 860 receives a focused image of a wide field of retina 52 including areas where a pattern laser treatment can be applied. An electronic display element 850 produces an overlay image that is reflected by beam-splitter 852 and overlaid together with the image of retina 52 focused onto image sensor 860.

Processor/controller 10 has real time position information of the light beam reflected by mirror 130 onto retina 52 as it controls the steering units 128 and 136. Processor/controller 10 also controls the pixel elements that compose display 850. Retinal imager 140 is precisely aligned with beam steering units 128 and 136 under processor/controller 10 command in a way that processor/controller 10 can display in display 850 an overlay image indicative of an area of retina 52 where a pattern of therapeutic laser treatment will be applied in response to a triggering action. Aiming display element 850 can be a high resolution mini-VGA panel such as Samsung TFT-LCD 640×480 pixels 1.98 inch display panel. Image sensor 860 can be the actual eye 618 of an operator observing through an eyepiece 616. Image sensor 860 can also be an electronic image sensor such as a CMOS or CCD image sensor capable of capturing an image of retina 52.

In cases where image sensor 860 is fixed in a way that the steering beams and the retinal image constantly aligned, then the aiming display element 850 can be omitted together with beam-splitter 852. In this situation processor/controller 10 can provide a virtual image with aiming information directly on a video display together with a retinal image sensor 860.

### Operation:

During a typical session using the steering beam therapeutic laser system 100 of the present invention, a first set of actions of processor/controller 10 is dedicated to initialization and calibration of actuators and sensors. The system remains idle waiting for user input through user interface 20. The operator must select a treatment pattern, the therapeutic laser power to be applied and a mode of operation i.e. repetitive or single burst modality. Secondary choices can be made such as the aiming beam pattern, treatment pattern sizes, orientation, spacing, etc.

Processor/controller 10 recalls data from memory 310 about the selected patterns and can recalculate new data according to pattern orientation and magnification or directly use the stored data. This data is related to various aspects of system 100 operation. A first action is to obtain the steering vectors to complete the selected treatment pattern path in the shortest time. A second action is to obtain the steering vectors to complete the selected aiming beam path during the interval system 100 is not delivering a burst of therapeutic laser energy. A third action if to obtain laser power modulation data for the path of the steering laser beam along the treatment area for to accurate delivery of the desired doses of therapeutic laser power onto the treatment locations of the pattern. A fourth action if to obtain aiming beam power modulation data for the path of the steering laser beam along retina 52 during the aiming process. Processor/controller 10 can obtain all these data by recalling it from memory 310 and/or by direct calculations at microcontroller 300 level. Processor/controller 10 can then store a table with the obtained data for the selected treatment pattern and for the selected aiming pattern, and further calculations can be made in real time during operation as required. Processor/controller 10 adjusts system 100 to perform according to operator selections such as laser beam size and laser beam profile.

Once the operator visualizes an area of retina 52 through retinal imager 140 an aiming image can be projected on the surface of retina 52, alternatively, the aiming image can be generated by processor/controller 10 as a video overlay from a display 850. The aiming image can also be generated by processor/controller 10 directly on a video monitor mixed with an image of retina 52. The aiming image can be displaced along the image of retina 52 viewed through retinal imager 140 using joystick 340. In detail, the operator moving joystick 340 provides voltage changes read as X-Y coordinates by processor/controller 10. A proportional output signal is produced by processor/controller 10 for operator XY steering mechanism 136 to steer the aiming image over the retinal image. In the preferred embodiment, an aiming beam pattern is projected onto the actual retina. In this case during the aiming process processor/controller 10 is rapidly steering and power modulating an aiming light. Simultaneously, beam steering mechanism 128 is used for the purpose of rapidly producing the aiming beam pattern that will be steered under operator command by operator XY steering mechanism 136. Fast operation of beam steering mechanism 128 provides steady visualization of aiming pattern because of retinal temporal integration of the operator's eye 618. Also fast operation of beam steering mechanism 128 provides safe delivery of the treatment pattern onto the retina during a burst of therapeutic laser energy.

Steering systems 128 and 136 can be integrated into a single wide field fast operating steering mechanism. The light used for producing the steering pattern can come from a separate visible light source 106 or from power laser source 102 attenuated by electronic, mechanic and/or optical means. Electronic attenuation can be obtained by applying a modulation signal from processor/controller 10 into power modulation input 202. Optical attenuation can be obtained by activating a subset of mirror elements of mirror array 800 under processor/controller 10 command.

In alternative embodiments where the aiming image is not actually projected onto the patient retina 52 but instead virtually produced at an overlay display or directly fed into a video monitor by processor/controller 10, optical alignment of components allows correspondence between the overlay steering image seen by an operator and the actual retinal locations of retina 52 permitting accurate therapeutic laser patterned beam positioning.

Once an operator has selected an area of retina 52 where a treatment pattern is desired a triggering action exerted through trigger 320 instructs processor/controller 10 to start a treatment sequence. A first optional action is to block operator aiming system 136 until the treatment pattern has been completed for enhanced accuracy. Processor/controller aligns beam steering mechanism 128 to direct the therapeutic laser beam to a starting location on retina 52 where delivery of the treatment pattern will be initiated.

Processor/controller 10 then performs a series of parallel actions for execution of one pattern of laser treatment. As a mode of example processor/controller 10 can: a) trigger therapeutic laser power ON and OFF along the pattern path using input 200, b) modulate the laser power between zero and a selected maximum power along the pattern path using input 202, c) regulate the speed of displacement of the steering laser beam at the retinal plane along the pattern path by driving unit 128. All these actions properly combined at programmatic level provide great flexibility to system 100. A single pattern can deliver spot shaped and/or linear shaped laser treatments onto therapeutic locations of retina 52. Treatment locations can receive laser power while the therapeutic laser beam is kept still by mechanism 128 or is displacing (“on the fly” treatment).

Once delivery of a single pattern of therapeutic laser energy has been completed under processor/controller 10 supervision, system 100 can return to an aiming mode of operation and can wait for a next operator triggering action. Alternatively, when a multi-burst mode of operation has been selected, once an timer interval has been completed, processor/controller 10 can automatically generate an internal triggering action to deliver of a new pattern of therapeutic laser energy. In this modality of operation, a series of treatment patterns can be automatically delivered as long as the operator is comfortable re-aiming the aiming pattern during the timer interval and sustains the initial triggering action.

The final output power delivered onto retina 52 per unit area can be adjusted by electronic or optical means. Electronic attenuation can be obtained by applying a modulation signal from processor/controller 10 into power modulation input 202 and/or into inhibit input 200. Optical attenuation can be obtained, for example, by activating mirror array 800 under processor/controller 10 control. Mirror array 800 is capable of switching ON and OFF each mirror element in 10 microseconds and has 307.200 individual mirrors. By having processor/controller 10 individually control the duty cycle of each mirror of the array, there is enormous flexibility to produce various schemes of amplitude and/or frequency modulated therapeutic laser beams. In situations where mirror array 800 is located in precise optical alignment inside laser delivery system 120, it can be further used to replace the functions of beam profiler 124 and/or of beam sizing unit 126, providing great flexibility for dynamic modulation of beam power, beam profile and beam size at periods below 2 microseconds (50 kHz). Mirror array 800 under processor/controller 10 command can also be used to attenuate the output of laser 102 to generate safe aiming light levels for the aiming pattern.

In embodiments where retinal imager 140 produces an image of an area of retina 52 onto a sensor 860 of electronic nature, processor/controller 10 can receive an input from imaging device 860 to add further processing power to protect sensitive zones of the retina in automatic fashion. For example a chromakey and/or a luminance key can be used under operator supervision to inhibit delivery of therapeutic laser energy to some treatment locations of a selected treatment pattern that would fall onto retinal locations that correspond with a retinal image of particular characteristics. For example, a mayor blood vessel, a scar, a pigmented zone, the macular area can all provide pixel characteristics to help processor/controller 10 block delivery of therapeutic laser energy onto these locations. In these embodiments, the operator would select luminance and/or chromakey laser block levels during the aiming process and observe on a video monitor the portions of the retinal image expected to be automatically excluded from receiving therapeutic laser energy. In the configuration where the retinal image is displayed on a video monitor, after-treatment effects can be monitored by processor/controller 10 using image analysis software to determining visual aspects of the treatment locations after a burst of laser is applied. Aspects related to the therapeutic effect such as color shift, size of affected area, etc. can provide useful feedback for treatment standardization. A series of audio feedback signals and visual signals are considered to alert an operator of the status of operation of system 100.

In further detail, methods for a procedure are described:

### A.-Manual Method: Steady Beam Treatment.

This method comprises the steps of:

1) selecting a treatment pattern.

2) selecting an area of the retina where a treatment pattern will be delivered using an aiming system,

3) having the operator deliver a START PATTERN SIGNAL to a controller system, for example by depressing a footswitch,

4) in response to said START PATTERN SIGNAL having the controller system:

4a) align the optics of the laser beam onto a first treatment location of said the pattern,

4b) activating the delivery of therapeutic laser energy to the retina during a programmed laser pulse duration setting,

4c) inactivating the delivery of therapeutic laser energy to the retina

4d) steering the optics of the laser beam onto a next treatment location of the pattern,

4e) activating the delivery of therapeutic laser energy to the retina during a programmed laser pulse duration setting,

4f) inactivating the delivery of therapeutic laser energy to the retina

5) repeating the steps of 4d), 4e) and 4f) until all the treatment locations of a selected pattern have been exposed to the therapeutic doses of laser energy.

6) selecting an area of the retina where a next treatment pattern can be delivered,

7) repeating the steps 3) to 6) until the selected treatment pattern has been applied to all the selected areas of the of the retina.

8) allowing the operator to end the delivery of therapeutic laser energy to the retina at any time, for example, by releasing a footswitch.

### B.-Semi-Automatic Method: Steady Beam Treatment.

A second method comprises the steps of:

1) selecting a treatment pattern

2) selecting an area of the retina where a treatment pattern will be delivered using an aiming system,

3) having the operator deliver a START SEQUENCE OF PATTERNS SIGNAL to a controller system, for example by depressing a footswitch.

4) in response to said START SEQUENCE OF PATTERNS SIGNAL having the controller system:

4a) automatically generating a START PATTERN SIGNAL

4b) align the optics of the laser beam onto a first treatment location of said the pattern,

4c) activating the delivery of therapeutic laser energy to the retina during a programmed laser pulse duration setting,

4d) inactivating the delivery of therapeutic laser energy to the retina

4e) steering the optics of the laser beam onto a next treatment location of the pattern,

4f) activating the delivery of therapeutic laser energy to the retina during a programmed laser pulse duration setting,

4g) inactivating the delivery of therapeutic laser energy to the retina

4h) repeating the steps of 4e), 4f) and 4g) until all the treatment locations of a selected pattern have been exposed to the therapeutic doses of laser energy.

4i) providing a predetermined interval to allow the operator to select an area of the retina where a next treatment pattern can be delivered,

4j) having the controller automatically generating a new START PATTERN SIGNAL,

4k) repeating the steps 4b) to 4j) until the operator provides an END SEQUENCE OF PATTERNS SIGNAL, for example by releasing a footswitch,

### C.-Manual Method: Flying Beam Treatment.

A third method comprises the steps of:

1) selecting a treatment pattern

2) selecting an area of the retina where a treatment pattern will be delivered using an aiming system,

3) having the operator deliver a START PATTERN SIGNAL to a controller system, for example by depressing a footswitch,

4) in response to said START PATTERN SIGNAL having the controller system:

4a) steering the optics of the laser beam to a starting position of a first treatment location of said the pattern,

4b) activating the delivery of therapeutic laser energy to the retina,

4c) steering the optics of the laser beam to an ending position of a first treatment location of said treatment pattern having therapeutic laser energy delivered to the treatment location while the activated therapeutic laser beam is in motion,

4d) inactivating the delivery of therapeutic laser energy to the retina

4e) steering the optics of the laser beam to a starting position of a next treatment location of the pattern,

4f) activating the delivery of therapeutic laser energy to the retina,

4g) steering the optics of the laser beam to an ending position of said next treatment location of said treatment pattern having therapeutic laser energy delivered to the treatment location while the activated therapeutic laser beam is in motion,

4h) inactivating the delivery of therapeutic laser energy to the retina

5) repeating the steps of 4e) to 4h) until all the treatment locations of a selected pattern have been exposed to the therapeutic doses of laser energy.

6) selecting an area of the retina where a next treatment pattern can be delivered,

7) repeating the steps 3) to 6) until the selected treatment pattern has been applied to all the selected areas of the of the retina.

8) allowing the operator to end the delivery of therapeutic laser energy to the retina at any time, for example, by releasing a footswitch.

### D.-Semi-Automatic Method: Flying Beam Treatment.

A fourth method comprises the steps of:

1) selecting a treatment pattern

2) selecting an area of the retina where a treatment pattern will be delivered using an aiming system,

3) having the operator deliver a START SEQUENCE OF PATTERNS SIGNAL to a controller system, for example by depressing a footswitch.

4) in response to said START SEQUENCE OF PATTERNS SIGNAL having the controller system:

4a) automatically generating a START PATTERN SIGNAL

4b) steering the optics of the laser beam to a starting position of a first treatment location of said the pattern,

4c) activating the delivery of therapeutic laser energy to the retina,

4d) steering the optics of the laser beam to an ending position of a first treatment location of said treatment pattern having therapeutic laser energy delivered to the treatment location while the activated therapeutic laser beam is in motion,

4e) inactivating the delivery of therapeutic laser energy to the retina

4f) steering the optics of the laser beam to a starting position of a next treatment location of the pattern,

4g) activating the delivery of therapeutic laser energy to the retina,

4h) steering the optics of the laser beam to an ending position of said next treatment location of said treatment pattern having therapeutic laser energy delivered to the treatment location while the activated therapeutic laser beam is in motion,

4i) inactivating the delivery of therapeutic laser energy to the retina

4j) providing a predetermined interval to allow the operator to select an area of the retina where a next treatment pattern can be delivered,

4k) having the controller automatically generating a new START PATTERN SIGNAL,

4l) repeating the steps 4b) to 4k) until the operator provides an END SEQUENCE OF PATTERNS SIGNAL, for example by releasing a footswitch,

### Steady Beam Treatment Computations:

When using a method that deliver pulses of therapeutic laser power with the laser beam aimed still over each treatment location of the retina, we can select the period that the therapeutic laser will be ON over that particular treatment location to deliver the desired laser energy. Adding all the periods of all the treatment locations in one pattern plus the steering time to move the beam between them gives the time required to deliver one full therapeutic laser pattern.

Factors such as retinal location, patient cooperation, modes of immobilizing the eye, operator preferences and experience can help to decide a therapeutic pattern with a pattern delivery time that is safe and comfortable for the operator to work with.

In general patterns with pattern delivery times less than 1000 milliseconds are preferred but longer lasting patterns can also be used under favorable conditions. Duration of each pulse can typically be set to between 5 and 1000 milliseconds, and this duration is interrelated with the therapeutic laser power setting and individual treatment location dimensions. Roughly single pulse duration is a function of the treatment location area divided by the laser power setting. Therapeutic laser pulses lasting between 10 and 50 milliseconds are preferred and it is practical to first set pulse duration and then adjust the laser power setting required to obtain the desired therapeutic effect for that pulse As a mode of example only, a hexagonal pattern of 19 treatment locations each receiving a dose of therapeutic laser energy lasting 15 milliseconds plus a settling time of 1 millisecond between 400 micron spaced locations at their center is typically laid down in about 0.3 seconds.

A standard retinal pan-photocoagulation procedure requiring 1800 treatment locations can be completed in less than 100 pattern applications with an effective treatment time under 30 seconds. Considering an average re-aiming time for an operator to reposition the pattern onto a new area of the retina between pattern applications of 1 second, the total re-aiming time is 100 seconds for said 100 pattern applications. Adding all treatment times, settling times and re-aiming times gives a total best case scenario pan-photocoagulation treatment duration of under 2 minutes, much less time than the one required for a similar pan-photocoagulation procedure using conventional single laser pulse methods.

### Flying Beam Treatment Computations:

When using a method that delivers doses of therapeutic laser energy to the retina while the laser beam is substantially in movement over the individual treatment locations that conform a single treatment pattern, the total time to treat a single pattern considers summing the time the laser is active traversing over each treatment location, plus the beam steering time between treatment locations. This method is more complex that a still beam method and requires high speed and computation power from processor/controller 10. In this modality the controller is programmed to make adjustments at least to the laser beam position and laser power in real time while it traverses over each treatment location of a predetermined treatment pattern. These adjustments may consider variation of the speed of the laser beam, of the energy delivered by the laser beam to the retina, of the size of the laser beam, and of the energy distribution pattern of the laser beam, to produce a desired therapeutic laser effect on each treatment location of a treatment pattern.

### CONCLUSION, RAMIFICATIONS AND SCOPE

While the above description contains many specificities these should not be construed as limitations on the scope of the invention, but rather as an exemplification of embodiments thereof. Accordingly, the scope of the invention should be determined not by the embodiments illustrated but by the appended claims and their legal equivalents.

1. A laser system for treating the retina, the system including:
a controller system,
an aiming system for an operator,
a therapeutic laser source and
a laser beam steering system,
in a way that said controller system commands said laser beam steering system to sequentially deliver therapeutic doses of laser energy from said therapeutic laser source to at least two treatment locations of said retina selected using said aiming system and in response to a single operator command

2. The system of claim 1, wherein said treatment locations includes at least one shape selected from the group consisting of spot shapes, linear shapes, symbol shapes or figure shapes.

3. The system of claim 1, further including a retinal imager for observing said retina.

4. The retinal imager of claim 3, wherein said retinal imager includes at least one component selected from the group consisting of a direct ophthalmoscope, an indirect ophthalmoscope, a scanning laser ophthalmoscope, a surgical microscope, a bio-microscope, a slit lamp, a retinal imaging contact lens, a non contact retinal video imager, a contact retinal video imager, a video display or an optical inverter.

5. The aiming system of claim 1 using aiming beam steering means to illuminate an area of the retina running over a perimeter line surrounding the peripheral treatment locations of a treatment pattern to be treated sequentially during a single burst of laser energy in response to a single operator command.

6. The aiming system of claim 1 using aiming beam steering means to illuminate areas of the retina approximately coincident with the peripheral treatment locations of a treatment pattern.

7. The aiming system of claim 1 using aiming beam steering means to illuminate areas of the retina approximately coincident with all the treatment locations of a treatment pattern

8. The aiming system of claim 1 using aiming beam steering means to illuminate areas of the retina coincident with all the treatment locations of a treatment pattern but substantially smaller.

9. The aiming system of claim 1 using aiming beam steering means to illuminate areas of the retina coincident with all the treatment locations of a treatment pattern but substantially bigger.

10. The aiming system of claim 1 using aiming beam steering means to illuminate an area of the retina approximately coincident with the area delineated by a perimeter line surrounding the most peripheral treatment locations of a treatment pattern.

11. The aiming system of claim 1 consisting in a virtual aiming image displayed as an overlay image by controller means using electronic display means optically aligned with an image of the retina seen through retinal imager means.

12. The aiming system of claim 1 consisting in a virtual aiming image displayed by controller means using video display means and mixed with a retinal image obtained using image sensor means through retinal imager means.

13. The system of claim 1, wherein the laser power delivered to said treatment locations during one burst of laser activity can be adjusted using laser power modulation means.

14. The system of claim 1, wherein the size of beam of laser power delivered to said treatment locations can vary using adjustable beam magnifier means.

15. The laser beam steering system of claim 1 including actuators selected from the group consisting of piezoelectric actuators, electrostatic actuators, MEMS based actuators, magnetostrictive actuators, voice-coil actuators, conventional motors and ultrasonic motors.

16. The system of claim 1, further including laser power modulator means based on an array of active micro-mirrors.

17. The system of claim 1 further including timer means adjustable to re-trigger the delivery of new sequences of therapeutic doses of laser energy at a preset interval without the need of a new operator command.

18. The system of claim 1 being capable of delivering therapeutic laser energy onto said treatment locations while said laser beam steering system is moving a beam of laser energy along said treatment locations.

19. A method for treating the retina with laser energy comprising:
a) selecting a therapeutic laser pattern with at least two locations;
b) observing the retina using retinal imaging means;
c) selecting an area of the retina to be treated using aiming means;
d) triggering one burst of said laser energy to sequentially deliver therapeutic doses of laser energy onto the retina according to said selected therapeutic laser pattern;
e) repeating steps b) to d) as required.

20. A method for treating the retina with laser energy comprising:
a) selecting a therapeutic laser pattern with at least two locations;
b) programming a timer interval
c) observing the retina using retinal imaging means;
d) selecting an area of the retina to be treated using aiming means;
e) triggering one burst of said laser energy to sequentially deliver therapeutic doses of laser energy onto the retina according to said selected therapeutic laser pattern;
f) activating said timer to start counting the programmed interval;
g) selecting another area of the retina to be treated using aiming means;
h) waiting for said timer interval to complete the programmed interval
i) having a controller system automatically trigger one burst of said laser energy to sequentially deliver therapeutic doses of laser energy onto the retina according to said selected therapeutic laser pattern;
k) repeating steps f) to i) as required.

21. A method to apply a pattern of therapeutic laser energy to at least two treatment locations of the retina in response to a single operator command comprising:
a) using image sensor means to detect an image of the retina where the pattern of therapeutic laser energy will be delivered;
b) using image analysis means to detect features on said image of the retina where therapeutic laser energy should not be delivered;
c) modifying said pattern of therapeutic laser energy to avoid delivery of therapeutic laser energy onto treatment locations that coincide with said detected features on said image of the retina, in a way that treatment locations of the retina that should not receive therapeutic laser energy are automatically protected from receiving laser treatment.

22. A method to modulate the power of a laser system for treating the retina comprising:
a) having a therapeutic laser beam aligned with the incident angle of a digitally operated mirror array;
b) having a light condenser element aligned with one reflection angle of said digitally operated mirror array;
c) having a light path receiving the output laser beam from said light condenser element;
d) having said light path deliver said laser beam reflected by said digitally operated mirror array onto said light condenser element using controllable means onto the retina;
e) having a mirror array controller selectively drive individual mirrors of said mirror array to reflect a fraction of the laser beam toward said light condenser element;

23. The method of claim 22 where said mirror array controller provides a frequency modulated laser beam by synchronously driving ON an OFF a plurality of mirror elements of said digitally operated mirror array.

24. The method of claim 22 where said mirror array controller provides an amplitude modulated laser beam by asynchronously driving ON an OFF the mirror elements of said digitally operated mirror array.
